(19) United States
(12) Patent Application Publication
(10) Pub. No.: US 2024/0366354 A1
(43) Pub. Date: Nov. 7, 2024

(54) ELECTRICALLY ACTUATED IMPLANTABLE CUFF

(71) Applicant: Boston Scientific Scimed, Inc., Maple Grove, MN (US)

(72) Inventor: James Ryan Mujwid, Hudson, WI (US)

(21) Appl. No.: 18/648,830

(22) Filed: Apr. 29, 2024

Related U.S. Application Data
(60) Provisional application No. 63/500,350, filed on May 5, 2023.

(51) Int. Cl. A61F 2/00

(52) U.S. Cl. CPC A61F 2/0036

(57) ABSTRACT

According to an aspect, an implantable device includes a frame member, and a tension member. The tension member has a first portion coupled to the frame member at a first location of the frame member and a second portion coupled to the frame member at a second location of the frame member. The tension member is configured to be disposed in a flexible state and a rigid state. The tension member is configured to be disposed in the rigid state when an electrical current is applied to the tension member.

### CROSS-REFERENCE TO RELATED APPLICATION

This application claims priority to U.S. Provisional Patent Application No. 63/500,350, filed on May 5, 2023, entitled “ELECTRICALLY ACTUATED IMPLANTABLE CUFF”, the disclosure of which is incorporated by reference herein in its entirety.

### TECHNICAL FIELD

This disclosure relates generally to an implantable device being configured to place pressure on a portion of a body of a patient, such as a urethra of the patient.

### BACKGROUND

In some medical devices, an implantable device is used to apply pressure to a portion of the body. For example, in some medical devices, the device is used to apply pressure to a urethra of a patient. Such devices may help treat incontinence.

Accordingly, there is a need for a medical device that may be inserted into the body of the patient and place pressure on the urethra of the patient to efficiently treat incontinence.

### SUMMARY

According to an aspect, an implantable device includes a frame member, and a tension member. The tension member has a first portion coupled to the frame member at a first location of the frame member and a second portion coupled to the frame member at a second location of the frame member. The tension member is configured to be disposed in a flexible state and a rigid state. The tension member is configured to be disposed in the rigid state when an electrical current is applied to the tension member.

In some implementations, the frame member is configured to be placed in an open configuration and a closed configuration. In some implementations, the frame member is configured to be placed in an open configuration and a closed configuration, the frame member being biased to the closed configuration. In some implementations, the frame member is configured to be placed in an open configuration and a closed configuration, the frame member being biased to the closed configuration, the frame member being configured to be placed in the open configuration in response to the tension member being placed in its rigid state.

In some implementations, the implantable device includes a control member operatively coupled to the tension member, the control member being configured to provide an electrical current to the tension member. In some implementations, the implantable device includes a control member operatively coupled to the tension member, the control member being configured to provide an electrical current to the tension member, the control member including a battery. In some implementations, the implantable device includes a control member, the control member being operatively coupled to the tension member via a wire. In some implementations, the implantable device includes a control member, a first wire extending from a first end portion of the tension member to the control member, a second wire extending from a second end portion of the tension member to the control member.

In some implementations, the frame member includes a first end portion and a second end portion, the first location of the frame member being disposed proximate the first end portion, the second location of the frame member being disposed proximate the second end portion. In some implementations, the frame member includes a first pivot and a second pivot, the frame member being configured to bend at the first pivot and bend at the second pivot. In some implementations, the frame member includes a first pivot and a second pivot, the frame member being configured to bend at the first pivot and bend at the second pivot, the tension member being slidable coupled to the first pivot and being slidably coupled to the second pivot.

In some implementations, the implantable device includes a housing, the housing containing the frame member and the tension member.

In some implementations, the tension member is a first tension member, the device includes a second tension member operatively coupled to the frame member. In some implementations, the tension member includes a nitinol material.

In some implementations, the frame member includes a non-conductive material.

According to another aspect, a method includes providing an implantable device comprising a frame member and a tension member, the tension member having a first portion coupled to the frame member at a first location of the frame member and a second portion coupled to the frame member at a second location of the frame member; and placing the implantable device within a body of a patient such that the implantable device is disposed adjacent at least a portion of a urethra of a patient.

In some implementations, the placing includes placing the implantable device within the body of the patient such that the implantable device surrounds the at least a portion of the urethra of the patient. In some implementations, the placing includes placing the implantable device within the body of the patient such that the implantable device is configured to place pressure on the at least a portion of the urethra of the patient.

In some implementations, the tension member is configured to be disposed in a flexible state and a rigid state, the tension member is configured to be disposed the rigid state when an electrical current is applied to the tension member.

In some implementations, the frame member is configured to be placed in an open configuration and a closed configuration, the frame member being biased to the closed configuration, the frame member being configured to be placed in the open configuration in response to the tension member being placed in the rigid state.

### BRIEF DESCRIPTION OF THE DRAWINGS

FIG. 1 schematically illustrates an implantable device according to an aspect.

FIG. 2 illustrates an implantable device according to another aspect disposed within a body of a patient.

FIG. 3 illustrates a portion of the implantable device of FIG. 2 disposed within the body of the patient.

FIG. 4 is a side view of a portion of the implantable device of FIG. 2 in a closed configuration.

FIG. 5 is a side view of a portion of the implantable device of FIG. 2 in an open configuration.

FIG. 6 is a cross-sectional view of a portion of an implantable device according to another aspect.

FIG. 7 side view of a portion of an implantable device according to another aspect.

FIG. 8 is a flow chart of a method according to an aspect.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, devices that are configured to be implanted or placed within a body of a patient are disclosed. An implantable device may be configured to be placed within a body of a patient and apply pressure to a portion of the body of the patient. For example, an implantable device may be configured to be placed within the body of a patient and apply pressure to a tubular portion of the body such as a urethra. In such cases, the implantable device may be placed within the body to help address incontinence issues.

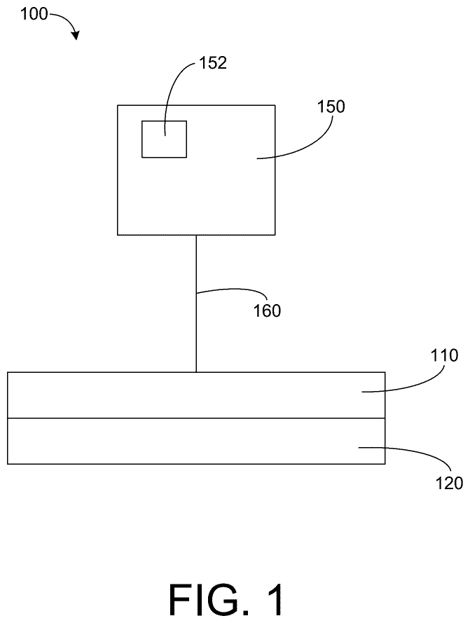

FIG. 1 schematically illustrates an implantable device 100 according to an aspect. In some implementations, the implantable device 100 is configured to be placed within a body of a patient and apply pressure to a portion of the body of the patient. For example, in some implementations, the implantable device 100 or a portion of the implantable device 100 is configured to be disposed proximate a urethra of a patient and is configured to apply or place pressure on a portion of the urethra of the patient. In other implementations, the implantable device 100 is configured to be placed at a different location within the body of the patient. In some implementations, the implantable device 100 or a portion of the implantable device forms a cuff configured to surround or partially surround a portion of the body of the patient.

The implantable device 100 includes a frame member 120, a tension member 110, and a control member 150. The tension member 110 is coupled to the frame member 120. For example, in some implementations, the tension member 110 is coupled to the frame member 120 at a variety or multiple locations or portion of the frame member 120. For example, in some implementations, the tension member 110 has a first end portion that is coupled to a first end portion of the frame member 120 and a second end portion that is coupled to a second end portion of the frame member 120. The tension member 110 may be fixedly coupled to the frame member 120 at some locations of the frame member 120 and may be slidably coupled to the frame member 120 at other locations of the frame member 120.

The tension member 110 is formed of a material that is configured to be placed in different configurations or states. For example, in some implementations, the tension member 110 is configured to be placed in a relaxed or flexible state and a stiff or rigid state. In some implementations, the tension member is larger when it is in the relaxed or flexible state and is configured to shrink or get smaller when it is in the stiff or rigid state. In some implementations, the tension member 110 is configured to assume its stiff or rigid state when an electrical current is applied to the tension member 110. In other words, in response to exposing the tension member 110 to an electrical current, the tension member 110 transitions from its relaxed and flexible state to its stiff and rigid state.

The frame member 120 is configured to be placed in a closed position or configuration and an open position or configuration. In the closed position or configuration, the frame member 120 is configured to apply a pressure to a portion of the body of the patient. For example, when the implantable device 100 is disposed proximate a urethra of a patient, the frame member 120 may be configured to apply pressure to (and help close) the urethra of the patient. The frame member 120 may be configured to release the pressure on the portion of the body of the patient (such as the urethra) when the frame member is in the open configuration or position.

In some implementations, the frame member 120 is biased to its closed position or configuration. In other words, at rest, the frame member 120 tends to assume its closed position or configuration. For example, in some implementations, the structure or the material of the frame member 120 serve to bias the frame member 120 into its closed position or configuration.

The tension member 110 is configured to engage and move the frame member 120 from its closed position or configuration to its open position or configuration. For example, when the tension member 110 is in its relaxed or flexible state, the frame member 120 is allowed to assume its closed position or configuration (the configuration to which the frame member 120 is biased). When the tension member 110 is in its stiff or rigid configuration, it is configured to overcome the bias of the frame member 120 and move the frame member 120 into its open position or configuration.

The control member or unit 150 is operatively coupled to the tension member 110. For example, the control member or unit 150 may be coupled to the tension member 110 via a wire. In the illustrated implementation, the control member or unit includes a power source 152 (such as a battery). The control member or unit 150 is configured to deliver current to the tension member 110. For example, in some implementations, the power source 152 is configured to deliver current to the tension member 110 via the wire 160.

In use, the implantable device 100 may be placed in the body of the patient such that the frame member 120 may apply pressure to a portion of the body of the patient. For example, the implantable device 100 may be placed within the body of the patient such that the frame member 120 is disposed adjacent to and configured to apply pressure to a urethra of the patient. In some implementations, the implantable device 100 is configured to be disposed within the body of the patient such that the frame member 120 surrounds or partially surrounds a portion of the urethra.

The frame member 120 is configured to apply pressure to the urethra while the frame member 120 is in its closed position or configuration. In some implementations, the pressure applied to the urethra of the patient helps close the urethra and helps treat urinary incontinence. In other implementations, the implantable device may be used to treat other types of incontinence.

When the patient needs to void, the patient may activate the control member 150 to apply an electrical current to the tension member 110. For example, the patient may use a Bluetooth or other device outside of the body to communicate with the control member 150 to activate the current. In response to the electrical current, the tension member 110 will assume its stiff or rigid state (and become smaller). This overcomes the biasing of the frame member 120 and causes the frame member 120 to move from its closed position or configuration to its open position or configuration. With the frame member 120 in its open position or configuration the pressure is at least partially removed from the urethra and the patient may void. Once the voiding is completed the current can be removed from the tension member 110. The tension member 110 will return to its relaxed or flexible state and the frame member 120 will return to its closed position or configuration (as it is biased to that configuration). Accordingly, pressure may then be fully applied to the urethra to help prevent unintentional voiding.

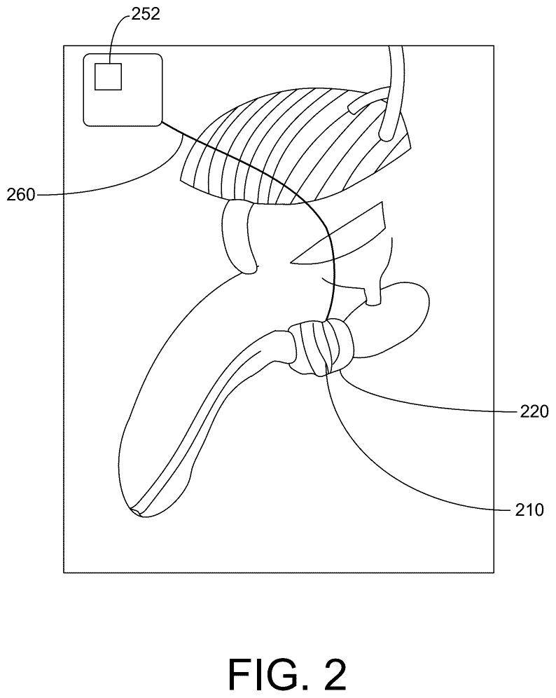

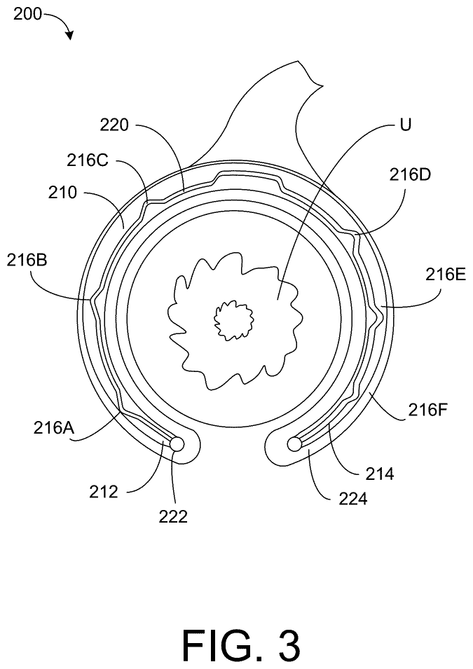

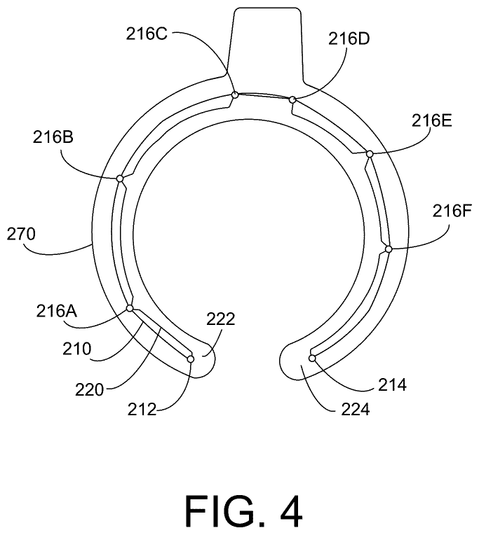

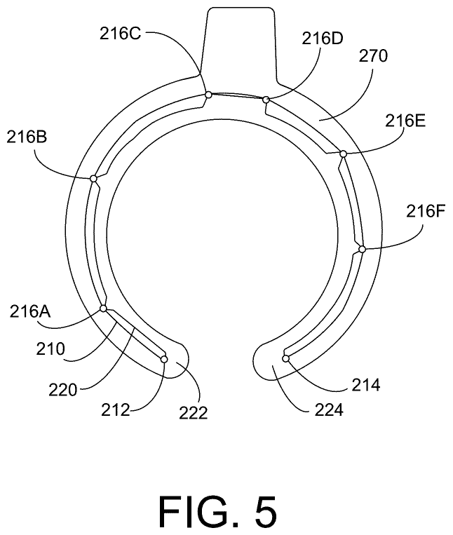

FIGS. 2-5 illustrate an implantable device 200 according to an aspect. FIGS. 2 and 3 illustrate the implantable device 200 disposed within a body of a patient. FIG. 4 is a side view of a portion of the implantable device 200 in a closed configuration. FIG. 5 is a side view of a portion of the implantable device 200 in an open configuration.

In the illustrated implementation, the implantable device 200 is configured to be placed within a body of a patient and apply pressure to a portion of the body of the patient. For example, as the implantable device 200 or a portion of the implantable device 200 is configured to be disposed proximate a urethra U of a patient and is configured to apply or place pressure on a portion of the urethra U of the patient. In other implementations, the implantable device 200 is configured to be placed at a different location within the body of the patient.

The implantable device 200 includes a frame member 220, a tension member 210, and a control member 250. The tension member 210 is coupled to the frame member 220. The tension member 210 is fixedly coupled to the frame member 220 at some locations of the frame member 220 and is slidably coupled to the frame member 220 at other locations of the frame member 220. Specifically, a first end portion 212 of the tension member 210 is fixedly coupled to a first end portion 222 of the frame member 220. A second end portion 214 of the tension member 210 is fixedly coupled to a second end portion 224 of the frame member 220. The tension member 210 is also slidably coupled to the frame member 220 at various locations, such as 216A, 216B, 216C, 216D, 216E, and 216F.

The tension member 210 is formed of a material that is configured to be placed in different configurations or states. For example, in some implementations, the tension member 210 is configured to be placed in a relaxed or flexible state and a stiff or rigid state. In some implementations, the tension member 210 is larger when it is in the relaxed or flexible state and is configured to shrink or get smaller when it is in the stiff or rigid state. In some implementations, the tension member 210 is configured to assume its stiff or rigid state when an electrical current is applied to the tension member 210. In other words, in response to exposing the tension member 210 to an electrical current, the tension member 210 transitions from its relaxed and flexible state to its stiff and rigid state.

In some implementations, the tension member 210 is formed of a nitinol material. In other implementations, the tension member 210 is formed of different materials. While a single tension member 210 is illustrated, in some implementations, the implantable device 200 includes more than one tension member. For example, in some implementations, the implantable device 200 includes two tension members.

The frame member 220 is configured to be placed in a closed position or configuration and an open position or configuration. In the closed position or configuration, the frame member 220 is configured to apply a pressure to a portion of the body of the patient. For example, when the implantable device 200 is disposed proximate a urethra U of a patient, the frame member 220 may be configured to apply pressure to (and help close) the urethra U of the patient. The frame member 220 may be configured to release the pressure on the portion of the body of the patient (such as the urethra) when the frame member is in the open configuration or position.

In some implementations, the frame member 220 is biased to its closed position or configuration. In other words, at rest, the frame member 220 tends to assume its closed position or configuration. For example, in some implementations, the structure or the material of the frame member 220 serve to bias the frame member 220 into its closed position or configuration.

The tension member 210 is configured to engage and move the frame member 220 from its closed position or configuration to its open position or configuration. For example, when the tension member 210 is in its relaxed or flexible state, the frame member 220 is allowed to assume its closed position or configuration (the configuration to which the frame member 220 is biased). When the tension member 210 is in its stiff or rigid configuration, the tension member 210 is configured to overcome the bias of the frame member 220 and move the frame member 220 into its open position or configuration.

In the illustrated implementation, the device 200 includes a housing 270. The housing 270 houses at least a portion of the frame member 220 and at least a portion of the tension member 210. The housing 270 may be formed of any biocompatible material. In some implementations, the housing 270 is formed of a silicone material.

The control member or unit 250 is operatively coupled to the tension member 210. For example, the control member or unit 250 may be coupled to the tension member 210 via a wire. In the illustrated implementation, the control member or unit includes a power source 252 (such as a battery). The control member or unit 250 is configured to deliver current to the tension member 210. For example, in some implementations, the battery 252 is configured to deliver current to the tension member 210 via the wire 260. In some implementations, the battery 252 is coupled to a portion (such as an end portion) of the tension member 210 via a first wire and is coupled to a second portion (such as a second end portion) of the tension member 210 via a second wire. The battery 252 is configured to deliver direct current to the tension member 210.

In use, the implantable device 200 may be placed in the body of the patient such that the frame member 220 may apply pressure to a portion of the body of the patient. For example, the implantable device 200 may be placed within the body of the patient such that the frame member 220 is disposed adjacent to and configured to apply pressure to a urethra U of the patient. In some implementations, the implantable device 200 is configured to be disposed within the body of the patient such that the frame member 220 surrounds or partially surrounds a portion of the urethra U.

The frame member 220 is configured to apply pressure to the urethra while the frame member 220 is in its closed position or configuration. In some implementations, the pressure applied to the urethra U of the patient helps close the urethra U and helps treat urinary incontinence. In other implementations, the implantable device 200 may be used to treat other types of incontinence.

When the patient needs to void, the patient may activate the control member 250 to apply an electrical current to the tension member 110. For example, the patient may use a Bluetooth or other device outside of the body to communicate with the control member 250 to activate the current. In response to the electrical current, the tension member 210 will assume its stiff or rigid state (and become smaller). This overcomes the biasing of the frame member 220 and causes the frame member 220 to move from its closed position or configuration to its open position or configuration. With the frame member 220 in its open position or configuration the pressure is at least partially removed from the urethra U and the patient may void. Once the voiding is completed the current can be removed from the tension member 210. The tension member 210 will return to its relaxed or flexible state and the frame member 220 will return to its closed position or configuration (as it is biased to that configuration). Accordingly, pressure may then be fully applied to the urethra U to help prevent unintentional voiding.

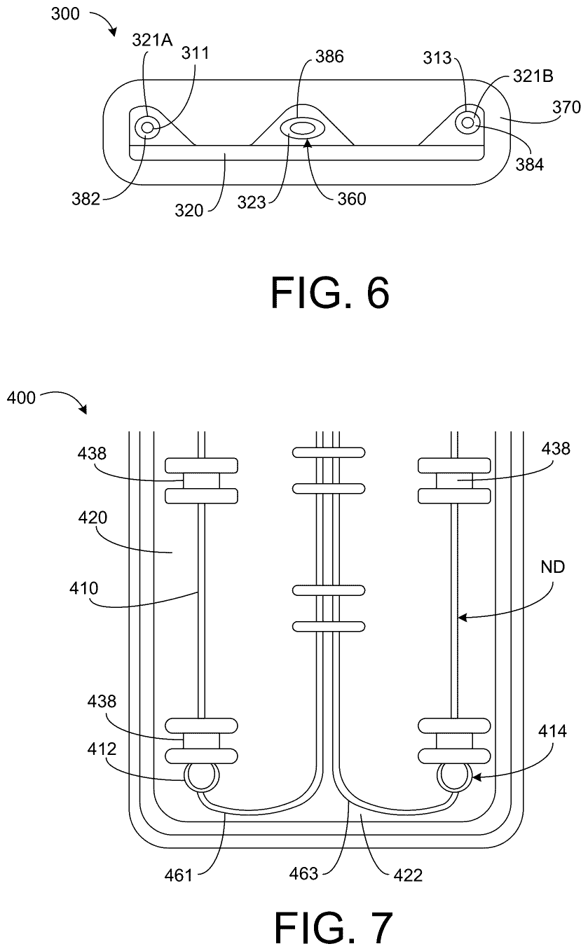

FIG. 6 is a cross-sectional view of a portion of an implantable device 300 according to an aspect. The implantable device 300 includes a frame member 320, a first tension member 311, a second tension member 313, and a wire 360 that operatively couples the tension members 311 and 313 to a control member (not illustrated). The tension members 311 and 313 extend along opposite side portions of the frame member 320. The frame member defines openings 321A and 321B. The tension member 311 extends through opening 321A and the tension member 313 extends through opening 321B. Accordingly, the tension members 311 and 313 are each slidable coupled to the frame member 320 at the opening 321A and 321B.

The frame member 320 also defines opening 333. The wire 360 extends through the opening 323. Accordingly, the wire 360 stays disposed apart from the tension members 311 and 313 at the mid-locations of the tension members 311 and 313.

In the illustrated implementation, the implantable device 300 includes insulators 382, 384, and 386. The insulators 382, 384, and 386 are disposed in the openings 321A, 321B, and 323. The insulators 382, 384, and 386 are configured to insulate or separate the tension members 311 and 313 and the wire 360 from the frame member 320. In such an implementation, the frame member 320 may be formed of a metal material.

In the illustrated implementation, the device 300 also includes a housing 370. The housing 370 houses at least a portion of the frame member 320 and at least a portion of the tension members 311 and 313. The housing 370 may be formed of any biocompatible material. In some implementations, the housing 370 is formed of a silicone material.

FIG. 7 is a cross-sectional view of an end portion of an implantable device 400 according to an aspect. The implantable device 400 includes a frame member 420, a tension member 410, and wires 461 and 463 that operatively couple the tension member 410 to a control member (not illustrated). The tension member 410 extends along a first side portion and a second side portion of the frame member 420. Specifically, as illustrated, the tension member 410 has a first end portion 412 that is coupled to the first end portion 422 of the frame member 420 and a second end portion 414 that is coupled to the first end portion 422 of the frame member 420. The tension member 410 forms a loop or U-shape at the second end of the frame member (not illustrated).

The frame member 420 defines guides 438 that slidably couple the tension member 310 to the frame member 420. In some implementations, the guides 438 also function as insulators to insulate or separate the tension member 310 from the frame member 420.

Wire 461 is coupled to the first end portion 412 of the tension member 410 and wire 463 is coupled to the second end portion 414 of the tension member 410. The wires 461 and 463 are configured to provide electrical current to the tension member 410. For example, in some implementations, the wire 461 may be operatively coupled to a positive electrode and the wire 463 may be operatively coupled to a negative electrode.

The tension members 311 and 313 extend along opposite side portions of the frame member 320. The frame member defines openings 321A and 321B. The tension member 311 extends through opening 321A and the tension member 313 extends through opening 321B. Accordingly, the tension members 311 and 313 are each slidably coupled to the frame member 320 at the opening 321A and 321B.

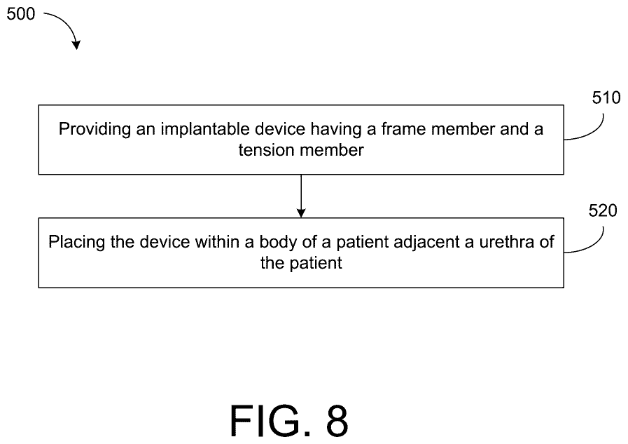

FIG. 8 is a flow chart of a method 500 according to an implementation. At 510, an implantable device is provided. The implantable device may be as the devices described above and include a frame member and a tension member. At 520, the implantable device is placed within or implanted into a body of a patient. In some implementations, the implantable device is placed within a pelvic region of a patient. For example, in some implementations, the implantable device is disposed proximate or adjacent a urethra of the patient. In some implementations, the implantable device is placed within the body such that the frame member surrounds a portion of the urethra of the patient. For example, in some implementations, the implantable device is placed such that the frame member forms a C shape around a portion of the urethra of the patient.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An implantable device comprising:
a frame member; and
a tension member, the tension member having a first portion coupled to the frame member at a first location of the frame member and a second portion coupled to the frame member at a second location of the frame member, the tension member being configured to be disposed in a flexible state and a rigid state, the tension member being configured to be disposed in the rigid state when an electrical current is applied to the tension member.

2. The implantable device of claim 1, wherein the frame member is configured to be placed in an open configuration and a closed configuration.

3. The implantable device of claim 1, wherein the frame member is configured to be placed in an open configuration and a closed configuration, the frame member being biased to the closed configuration.

4. The implantable device of claim 1, wherein the frame member is configured to be placed in an open configuration and a closed configuration, the frame member being biased to the closed configuration, the frame member being configured to be placed in the open configuration in response to the tension member being placed in its rigid state.

5. The implantable device of claim 1, further comprising:
a control member operatively coupled to the tension member, the control member being configured to provide an electrical current to the tension member.

6. The implantable device of claim 1, further comprising:
a control member operatively coupled to the tension member, the control member being configured to provide an electrical current to the tension member, the control member including a battery.

7. The implantable device of claim 1, further comprising:
a control member, the control member being operatively coupled to the tension member via a wire.

8. The implantable device of claim 1, further comprising:
a control member, a first wire extending from a first end portion of the tension member to the control member, a second wire extending from a second end portion of the tension member to the control member.

9. The implantable device of claim 1, wherein the frame member includes a first end portion and a second end portion, the first location of the frame member being disposed proximate the first end portion, the second location of the frame member being disposed proximate the second end portion.

10. The implantable device of claim 1, wherein the frame member includes a first pivot and a second pivot, the frame member being configured to bend at the first pivot and bend at the second pivot.

11. The implantable device of claim 1, wherein the frame member includes a first pivot and a second pivot, the frame member being configured to bend at the first pivot and bend at the second pivot, the tension member being slidable coupled to the first pivot and being slidably coupled to the second pivot.

12. The implantable device of claim 1, further comprising:
a housing, the housing containing the frame member and the tension member.

13. The implantable device of claim 1, wherein the tension member is a first tension member, the device further comprising:
a second tension member operatively coupled to the frame member.

14. The implantable device of claim 1, wherein the tension member includes a nitinol material.

15. The implantable device of claim 1, wherein the frame member includes a non-conductive material.

16. A method, comprising:
providing an implantable device comprising a frame member and a tension member, the tension member having a first portion coupled to the frame member at a first location of the frame member and a second portion coupled to the frame member at a second location of the frame member; and
placing the implantable device within a body of a patient such that the implantable device is disposed adjacent at least a portion of a urethra of a patient.

17. The method of claim 16, wherein the placing includes placing the implantable device within the body of the patient such that the implantable device surrounds the at least a portion of the urethra of the patient.

18. The method of claim 16, wherein the placing includes placing the implantable device within the body of the patient such that the implantable device is configured to place pressure on the at least a portion of the urethra of the patient.

19. The method of claim 16, wherein the tension member is configured to be disposed in a flexible state and a rigid state, the tension member is configured to be disposed the rigid state when an electrical current is applied to the tension member.

20. The method of claim 19, wherein the frame member is configured to be placed in an open configuration and a closed configuration, the frame member being biased to the closed configuration, the frame member being configured to be placed in the open configuration in response to the tension member being placed in the rigid state.
